(19) United States
(12) Patent Application Publication
(10) Pub. No.: US 2006/0031021 A1
(43) Pub. Date: Feb. 9, 2006

(54) In vitro predictive method

(75) Inventors: Jaymin C. Shah, Waterford, CT (US); Agnieszka D. Machate, Waterford, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/195,894

(22) Filed: Oct. 24, 2005

Related U.S. Application Data
(63) Continuation of application No. PCT/IB05/01417, filed on May 13, 2005.
(60) Provisional application No. 60/574,637, filed on May 26, 2004.

(51) Int. Cl. G01N 33/48

(52) U.S. Cl. 702/19

(57) ABSTRACT

An in vitro method for predicting in vivo pharmacokinetic parameters such as Cₘₐₓ for poorly soluble drug compounds in formulations.

This invention claims priority and is a Continuation application under 35 U.S.C. 120 of PCT/IB2005/001417, filed May 13, 2005 and designating the United States, which claims priority under 35 U.S.C. 119(e) of U.S. Ser. No. 60/574,637, filed May 26, 2004.

### FIELD OF THE INVENTION

The invention pertains to in vitro methods of predicting in vivo pharmacokinetic (PK) parameters, e.g. Cₘₐₓ. The invention also relates to pharmacokinetic properties of poorly soluble drugs, such as ziprasidone, and to depot formulations comprising same.

### BACKGROUND OF THE INVENTION

The development of drug compounds and formulations requires methods to predict performance and behavior. Among these are dissolution tests by which in vitro-in vivo correlations (IVIVC) are made. Traditionally, in vitro dissolution methods under so-called sink conditions have been used to develop and screen compounds and formulation concepts, including controlled release formulations such as intramuscular (IM) depot formulations. Sink conditions generally refer to the circumstances wherein the amount of drug compound or formulation that can be dissolved in a dissolution medium is 5 to 10× the amount of drug to be dissolved.

While widely used, sink methods have limitations, working best for soluble compounds and formulations. Poorly soluble compounds however are not amenable to sink techniques: their low solubility makes it difficult to achieve sink conditions in the first instance, including, importantly, conditions that simulate the injection site; indeed, these conventional dissolution techniques have proven to be either unworkable or non-predictive where poorly soluble drugs are concerned. This is true, for example, with compounds such as ziprasidone and the development of depot formulations containing same.

Ziprasidone is a chlorooxyindole class of aryl-heterocyclic compound having psychotropic effect; it is an atypical anti-psychotic often prescribed for treating schizophrenia. Because non-compliance with such medication is a pressing problem in treating this disease, long acting dosage forms which minimize the need for patient self-administration are desirable. Among these are depot formulations, especially injectables, which provide slow absorption of the drug from the site of administration. Because the solubility of its most soluble salt (ziprasidone mesylate) is low, dissolution testing using sink conditions to further develop and refine such formulations by e.g. being able to reliably perform IVIVCs so to predict pharmacokinetics, is entirely unsuitable.

Hence a need for an in vitro method to predict in vivo pharmacokinetics of poorly soluble drug compounds such as ziprasidone and formulations containing same subsists.

### SUMMARY OF THE INVENTION

The invention addresses the foregoing need. In one practice, the invention is an in vitro method for predicting in vivo pharmacokinetics of a poorly soluble drug compound in a test formulation which comprises a) contacting said test formulation with a liquid release medium under conditions effective to form a precipitate and a supernatant; b) determining the concentration of said drug compound in said supernatant; and c) correlating said concentration to at least one in vivo pharmacokinetic parameter to predict same for said test formulation. Preferably, for step (c), said in vivo pharmacokinetic parameter to which correlation is made is derived from a pre-established profile in an animal model using said poorly soluble drug compound in one or more formulations that are different than said test formulation. In one embodiment the correlating step (c) involves linear regression analysis.

In one practice, said drug compound is an aryl-heterocyclic compound, preferably solubilized or in suspension. For example, in one embodiment, said aryl-heterocyclic compound is ziprasidone, preferably solubilized with a cyclodextrin such as e.g. γ-cyclodextrin, β-cyclodextrin, HPBCD, SBECD or mixtures thereof; and/or the ziprasidone can be in suspension with a viscosity agent such as e.g. a celluose derivative, polyvinylpyrrolidone, alginates, chitosan, a dextrin, gelatin, polyethylene glycols, polyoxyethylene ethers, polyoxypropylene ethers, polyesters, polylactides, polyglycolides, polycaprolactones, polyanhydrides, polyamines, polyurethanes, polyesteramides, polyorthoesters, polydioxanes, polyacetals, polycarbonates, polyorthocarbonates, polyphosphazenes, succinates, polyorthocarbonates, poly(maleic acid), poly(amino acids), polyhydrocellulose, chitin, copolymers or terpolymers of the foregoing, sucrose acetate, isobutyrate, PLGA, stearic acid/NMP, or any combination of the foregoing.

In a preferred practice, the liquid release medium has a pH, ionic strength, buffer capacity and/or temperature similar to an in vivo injection site, e.g. wherein said pH is about 7.4 and said temperature is about 37° C. In one practice, the liquid release medium comprises a physiological buffer, which optionally can comprise gel or albumin.

The in vivo pharmacokinetic parameter that can be predicted includes Cₘₐₓ or depot level or both. In a particularly preferred embodiment, the invention is directed to a non-sink in vitro method for predicting in vivo pharmacokinetics of a depot test formulation containing a poorly soluble drug compound, e.g. ziprasidone, which comprises a) contacting said depot test formulation with a liquid release medium comprising a physiological buffer having a pH of about 7.4 at a temperature of about 37° C. under conditions effective to form a precipitate and a supernatant; b) determining the concentration of said poorly soluble drug compound in said supernatant; and c) correlating said concentration to Cₘₐₓ or depot level to predict same in vivo for said depot test formulation. Correlation can be done using pre-established animal profiles as explicated herein.

### BRIEF DESCRIPTION OF THE DRAWINGS

FIGS. 1 and 2 show correlation between C_{depot} (in vivo, dog) and in vitro C_{24 hrs} (FIG. 1) and in vitro C_{7 days} (FIG. 2) obtained in practicing the invention.

FIGS. 3 and 4 show correlation between Cₘₐₓ (in vivo, dog) versus in vitro C_{15 min} (FIG. 3) and in vitro C_{1 hr} (FIG. 4) obtained in practicing the invention.

### DETAILED DESCRIPTION OF THE INVENTION

Poorly Soluble Drug Compounds:

The qualifier “poorly soluble” as applied herein to drug compounds is understood by those in the art. The term includes drug compounds considered insoluble. Without restriction, the term includes compounds having a solubility of about 1 mg/ml or less. Preferred compounds in this regard include aryl-heterocylics, preferably those having psychotropic effects, such as those of the chlorooxyidole class, most preferably ziprasidone. Without limitation, an embodiment of an aryl-heterocyclic compound subject to the practice of the present invention has the structure: 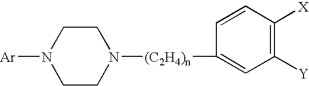
wherein
Ar is benzoisothiazolyl or an oxide or dioxide thereof, each optionally substituted by one fluoro, chloro, trifluoromethyl, methoxy, cyano, or nitro: n is 1 or 2; and
X and Y together with the phenyl to which they are attached form benzothiazolyl; 2-aminobenzothiazolyl; benzoisothiazolyl; indazolyl; 3-hydroxyindazolyl; indolyl; oxindolyl optionally substituted by one to three of (C₁-C₃) alkyl, or one of chloro, fluoro or phenyl, said phenyl optionally substituted by one chloro or fluoro; benzoxazolyl; 2-aminobenzoxazolyl; benzoxazolonyl; 2-aminobenxozazolinyl; benzothiazolonyl; benzoimidazolonyl; or benzotriazolyl. Representative examples of compounds falling within the foregoing definition are found in U.S. Pat. No. 4,831,031 incorporated herein by reference.

In one practice, the invention preferably applies to the above compounds wherein X and Y together with the phenyl to which they are attached form oxindole; more preferably, the oxindole moiety is 6-chlorooxindole-5-yl. In another preferred practice, Ar is benzoisothiazoyl; in still another preferred practice, n is 1. A particularly preferred aryl-heterocyclic to which the invention pertains is ziprasidone, 5-[2-[4-(1,2-benzisothiazol-3-yl)-1-piperazinyl]ethyl]-6-chloro-1,3-dihydro-2H-indol-2-one, which has the structure:

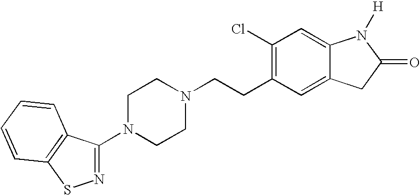

Although the aryl heterocyclic compound described herein may be constituted as a free base, it is preferred if aryl-heterocyclic compound is present as a pharmaceutically acceptable salt. The term “salt” in this regard intends pharmaceutically acceptable acid addition salts of aryl-heterocyclics, including ziprasidone. For purposes of preparing the kit or formulation of the invention, the salts can be anhydrous or in the form of one or more solvates, such as hydrates, including mixtures thereof. The salts may also occur in different polymorphic forms. By way of exemplification only, mesylate salts of the aryl heterocyclic ziprasidone may be present in dihydrate or trihydrate forms as disclosed in U.S. Pat. Nos. 6,110,918 and 6,245,765 both of which are incorporated herein by reference. Without limitation, preferred salts are selected from the group consisting of the tosylate, tartrate, hydrochloride, napsylate, besylate, aspartate, esylate and mesylate salt. In an especially preferred practice, the aryl heterocyclic is ziprasidone mesylate, more preferably in the trihydrate form.

Although the course of the following discussion is presented in terms of ziprasidone it will be apparent to the artisan that same is readily adapted to other poorly soluble compounds as contemplated by the invention.

The term “mgA” as in e.g. “mgA/ml” as used herein relates to the weight (in mg) of drug compound, e.g. ziprasidone, calculated as free base; for ziprasidone, molecular weight=412.9.

Techniques to solubilize ziprasidone to increase levels of concentration are known in the art and involve, without limitation, the use of cyclodextrins and other solubilizers.

A preferred solubilizer is a cyclodextrin. Cyclodextrins are cyclic oligosaccharides with hydroxyl groups on the outer surface and a void cavity in the center. The outer surface is usually hydrophilic hence cyclodextrins are soluble in water. The void on the other hand is typically hydrophobic. Cyclodextrins have the ability to form complexes with guest molecules, such as ziprasidone. Cyclodextrins contemplated by the invention include without limitation: α, β, γ-cyclodextrins, methylated cyclodextrins, hydroxypropyl-β-cyclodextrin (HPBCD), hydroxyethyl-β-cyclodextrin (HEBCD), branched cyclodextrins in which one or two glucoses or maltoses are enzymatically attached to the cyclodextrin ring, ethyl- and ethyl-carboxymethyl cyclodextrins, dihydropropyl cyclodextrins, and sulfoalkyl ether cyclodextrins, such as sulfobutyl ether-β-cyclodextrin (SBECD). The cyclodextrins can be unsubstituted or substituted in whole or in part as known in the art; mixtures of cyclodextrins are also useable. The preferred cyclodextrins for a typical depot formulation include β-cyclodextrin, HPBCD, SBECD or mixtures thereof; SBECD being most preferred.

Cyclodextrin complexes with ziprasidone can be rendered soluble in water as described in U.S. Pat. No. 6,232,304 incorporated by reference above.

Alternatively or conjointly, the ziprasidone may also be in the form of a suspension. Such formulations may also include viscosity agents as known in the art, e.g. viscosified water, pharmaceutically acceptable oils and oil-based agents, polymeric agents and other non-aqueous viscous vehicles. Preferred viscosity agents include without limitation: cellulose derivatives, polyvinylpyrrolidone, alginates, chitosan, dextrans, gelatin, polyethylene glycols, polyoxyethylene ethers, polyoxypropylene ethers, polyesters, polylactides, polyglycolides, polycaprolactones, polyanhydrides, polyamines, polyurethanes, polyesteramides, polyorthoesters, polydioxanones, polyacetals, polycarbonates, polyorthocarbonates, polyphosphazenes, succinates, polycarbonates, poly(maleic acid), poly(amino acids), polyhydroxycellulose, chitin, copolymers and terpolymers of the foregoing, and mixtures thereof. Preferred cellulose derivatives include methyl cellulose, sodium carboxymethyl celluose (NaCMC) and hydroxypropyl methyl cellulose. Preferred polylactides, polyglycolides, copolymers and terploymers thereof include poly-lactic-co-glycolic acid (PLGA). Also useful as viscosity agents are in situ gelling systems, e.g. stearic acid (SA) and NMP combinations, sucrose acetate isobutyrate and PLGA.

Injectable depot formulations are those effective for treatment of illnesses such as schizophrenia over a sustained period of time, i.e. for a period of time beyond that which is obtained by immediate release injection systems. Thus by way of further definition an injectable depot formulation provides, for example, efficacious plasma levels of active agent for at least 8 hours using typical injection volumes, e.g. about 0.1 ml to about 3 ml., about 1 ml to about 2 ml being usual. Preferably, the sustained period provided by the invention is at least 24 hours; more preferably up to about 1 week; still more preferably from about 1 week to about 2 weeks or more including up to about 8 weeks using the injection volumes aforesaid. For example, in the case of ziprasidone, a depot formulation can deliver at least 1 to about 420 mgA in an injection volume of about 1-2 ml for about 1 to about 2 weeks or more, including up to about 8 weeks. More preferably, about 10 to about 210 mgA for up to about 2 weeks.

Liquid Release Medium:

Liquid release media suitable for the present invention preferably include those simulative of in vivo injection sites, especially IM injection sites. In vivo as used herein refers to the class Mammalia, including, representatively, dogs, cats and humans. Without limitation in this regard, it is preferred if the liquid release medium mimic one or more of the following of an in vivo IM injection site: pH, ionic strength, buffer capacity and/or temperature. For example, whereas pH can be about 1 to about 8, it is preferred that it be about 7.4. Preferred temperature of the medium is between about 34° to 40° C., more preferably about 37° C. In one embodiment, the liquid release medium comprises a physiological buffer solution (PBS) as defined herein, or as otherwise known in the art. Said physiological buffer may be gelled or contain proteinacious material such as plasma proteins, e.g. albumin, and the like. Preferred liquid media are PBS and albumin-containing-PBS.

Contact of the formulation containing said poorly soluble drug compound with the liquid release medium may be accomplished by methods known in the art, including injection. Without limitation, contact of such formulation, especially a depot formulation, with a physiological buffer at a pH of about 7.4 and a temperature of about 37° C. in the practice of the invention results in the formation of a precipitate and a supernatant. Formation of the precipitate and supernatant in accordance with the invention is referred to herein as a non-sink condition or method. Other media mimicking in vivo conditions can be envisaged by those of ordinary skill in the art and may be employed to effectuate the precipitate/supernatant non-sink condition on contact; all such media are contemplated as within the invention.

PK Correlations:

PK parameters predictable by the present invention include those employed in the ordinary course of drug development. Without limitation, these include Cₘₐₓ and C_{depot}. The common understanding of these terms by the artisan is applicable herein. By way of example only in this regard: Cₘₐₓ is typically the maximum concentration of drug measured in serum (e.g. blood) after administration. The time it takes to reach Cₘₐₓ is denoted tₘₐₓ; for example, in an embodiment of the invention Cₘₐₓ for various depot formulations of ziprasidone is generally manifested in about 15 minutes to about 30 minutes. C_{depot} (depot level) is typically the average serum concentration between set time periods, e.g. the average concentration measured periodically between 12 hrs and 14 days.

In practice, the concentration of the drug compound in the supernatant is determined by means known in the art. Concentrations in this regard may be measured at one or more points in time, e.g. after 15 min, 1 hr, 24 hrs or up to about 7 days or more, e.g. 14 days. Concentration thus determined according to the present invention is correlated with various in vivo parameters aforesaid such as Cₘₐₓ and/C_{depot}.

Correlations serviceable for the invention can be obtained by any manner known to the art. By way of example only, correlations can be obtained by pre-establishing profiles for the pharmacokinetic parameters of concern (e.g. Cₘₐₓ, depot level) in suitable animal models (e.g. dogs) using one or more formulations comprising the poorly soluble drug compound of interest. The pre-established profiles can then be statistically assessed against the concentrations of the same formulations as measured in the supernatant of the inventive practice as aforesaid. Any statistical method can be utilized to compare the two data sets that result (pre-established and supernatant), e.g. linear regression analysis. In vivo performance of other formulations comprising the poorly soluble drug compound can thereafter be predicted by correlating the supernatant concentrations of same to the parameters determined as aforesaid.

The following examples are illustrative only. They are not limitative to the scope of the invention.

### EXAMPLES

Procedures:
1.) The following three media A, B and C (D was a control) simulating in vivo IM injection site with respect to pH, ionic strength, buffer capacity and temperature (37° C.) were employed:
A. Physiological buffer (PBS): 66.7 mM Phosphate buffer pH 7.3, Buffer Capacity calculated to be 0.037 (blood: 0.039), Ionic Strength calculated to be 0.206 (normal saline 0.154).
B. Gelled PBS: Physiological buffer (pH 7.5) gelled with 20% w/v pluronic F127 NF to simulate viscosity and consistency of the muscle tissue
C. Albumin in physiological buffer at 35 mg/ml, pH 7.3: Serum albumin levels range from 35 to 55 mg/ml to study the effect of protein binding on local drug distribution and precipitation
D. Phosphate buffer, pH 4.0 as a control: 33.4 mM monobasic sodium phosphate solution
2.) The following formulations containing ziprasidone (mesylate form) were evaluated in each of the above media: depot formulations B-E were true solutions (40 to 80 mgA/ml) utilizing either high concentration of SBECD; or a cooperative solubilization with cosolvent(s) such as n-methyl-pyrrolidone (NMP), dimethyl sulfoxide (DMSO), ethanol and the like, and SBECD (molar ratio refers to the ratio of ziprasidone mesylate to SBECD in the formulation):
A. Immediate release (control): ziprasidone at 20 mgA/ml with 30% SBECD
B. IM 1: ziprasidone at 40 mgA/ml in water with 56% SBECD, 0.42% NaCMC, and 40 mg PVP/ml.
C. IM 2: ziprasidone at 80 mgA/ml in water with 56% SBECD, 0.42% NaCMC, and 40 mg PVP/ml.
D. IM 3: ziprasidone at 80 mgA/ml in 34% NMP in water with 23% SBECD, 29% PEG 3350, and 40 mg PVP/ml.
E. IM 4: ziprasidone at 140 mgA/ml in 30% Benzyl Benzoate/70% Pyrrolidone with 40% SBECD.

Approximately 40% volume expansion may be expected for IM 2 and IM 3.
3.) Characterization of drug concentration in the liquid release medium
4.) Experimental Conditions
   A. Injection volume of each formulation: 0.5 ml
   B. Volume of Release Medium: 10 ml
   C. Temperature: 37° C. (incubator oven)
   D. Duration: 7 days
      a. Sampling time points: upon injection, after 15 minutes, at 1 hour and 24 hours, and after 1 week
      b. Precipitate separated from supernatant by means of centrifugation
   F. Supernatant preparation for HPLC Analysis
      a. The supernatant filtered through 0.22-μm membrane prior to analysis

In Vitro-In Vivo Correlation (IVIVC)

In vivo PK performance of these formulations using an in vitro method was established as follows: The subject formulations were dosed in dogs and entire PK profiles obtained. In vivo Cₘₐₓ and mean depot levels (e.g. average C_{12 hours} to C_{t last} levels, wherein C_{t last} is the concentration at the time of final measurement) were correlated with concentration of ziprasidone obtained in the release medium upon dosing with the same formulations. To establish the IVIVC, the Cₘₐₓ (burst at 15 minutes in vivo) was correlated with in vitro C_{15 minutes} and C_{1 hour}, and the mean depot levels (average C_{12 hours} to C_{t last} levels) were plotted against in vitro C_{24 hours} and C_{7 days}. In vitro data (pH 7.4) were employed in the IVIVC investigations. In vitro data were plotted as independent variables, and a correlation coefficient with the best-fit line was established for observed significant correlation.

IVIVC for Depot Levels

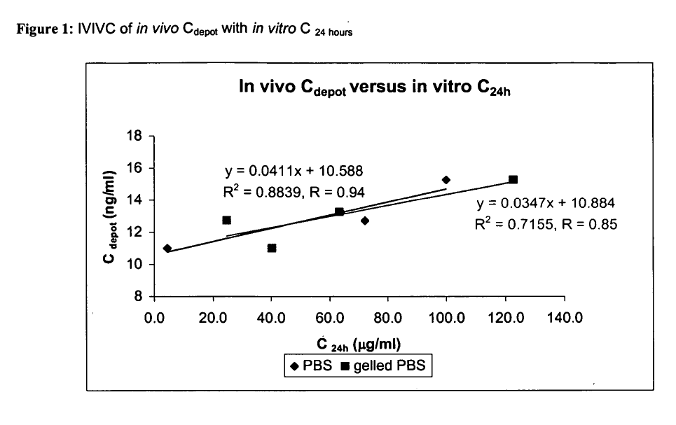

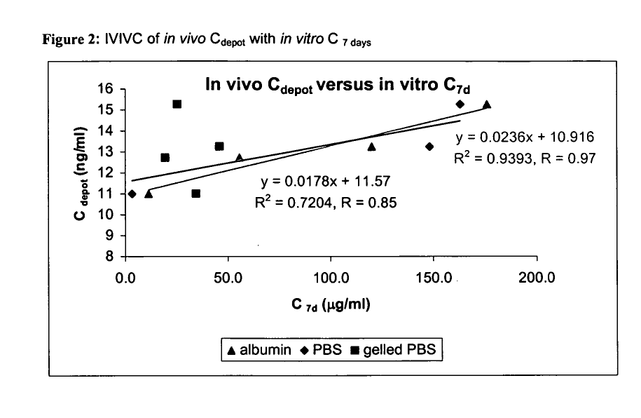

In one aspect, the in vitro method of the invention predicts depot levels and enables development and screening for formulations that result in higher depot levels in vivo. Mean depot levels (e.g. average of serum levels between C_{12 hours} to C_{t last}) observed in vivo were plotted against in vitro C_{24 hours} and C_{7 days} as shown in FIGS. 1 and 2 respectively:

A strong linear IVIVC was observed between depot levels and C_{24 hours} in PBS, C_{24 hours} in gelled PBS, C_{7 days} in PBS, and C_{7 days} in albumin-containing PBS as reflected by linear regression coefficient (R²) values of 0.88, 0.72, 0.72, and 0.94, respectively.

IVIVC for Burst or Cₘₐₓ Levels

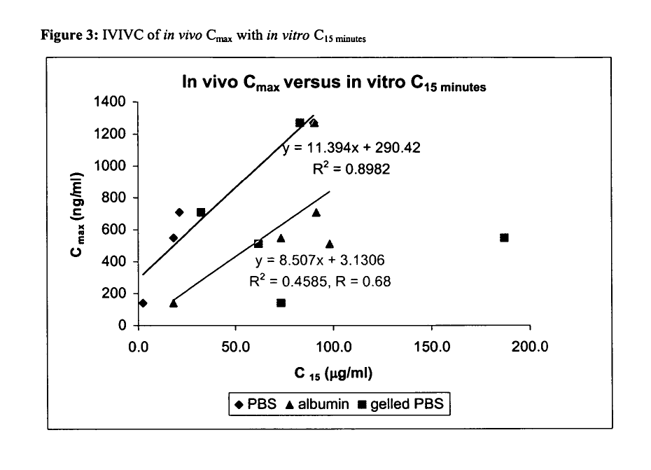

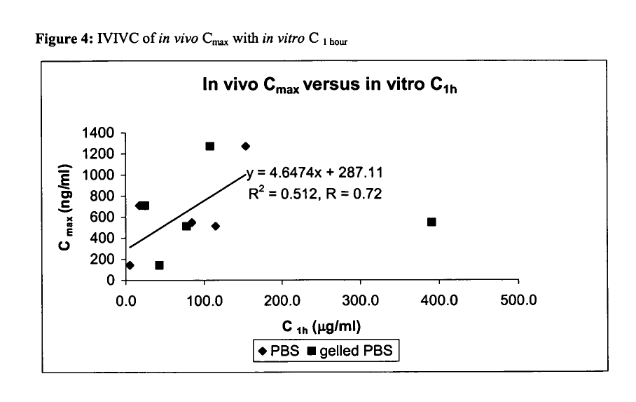

In vivo Cₘₐₓ is correlated with in vitro C_{15 minutes} in FIG. 3 and with in vitro C_{1 hour} in FIG. 4.

A strong correlation was observed for PBS as a medium with correlation coefficient (R) of 0.681. The in vitro C_{15 minutes} in PBS was thus a reliable predictor of in vivo Cₘₐₓ for these depot formulations.

1. An in vitro method for predicting in vivo pharmacokinetics of a poorly soluble drug compound in a test formulation which comprises:
a) contacting said test formulation with a liquid release medium under conditions effective to form a precipitate and a supernatant;
b) determining the concentration of said drug compound in said supernatant;
c) correlating said concentration to at least one in vivo pharamacokinetic parameter to predict same for said test formulation.

2. The method of claim 1 wherein, for step (c), said in vivo pharmacokinetic parameter to which correlation is made is derived from a pre-established profile in an animal model using said drug compound in one or more formulations different from said test formulation.

3. The method of claim 2 wherein said correlating involves linear regression analysis.

4. The method of claim 1 wherein said drug compound is an aryl-heterocyclic compound.

5. The method of claim 4 wherein said aryl-heterocyclic compound is solubilized or in suspension.

6. The method of claim 1 wherein said compound is solubilized with a cyclodextrin.

7. The method of claim 6 wherein said cyclodextrin is γ-cyclodextrin, β-cyclodextrin, HPBCD, SBECD or mixtures thereof.

8. The method of claim 1 wherein said compound is in suspension with a viscosity agent.

9. The method of claim 8 wherein said viscosity agent comprises a celluose derivative, polyvinylpyrrolidone, alginates, chitosan, a dextrin, gelatin, polyethylene glycols, polyoxyethylene ethers, polyoxypropylene ethers, polyesters, polylactides, polyglycolides, polycaprolactones, polyanhydrides, polyamines, polyurethanes, polyesteramides, polyorthoesters, polydioxanes, polyacetals, polycarbonates, polyorthocarbonates, polyphosphazenes, succinates, polyorthocarbonates, poly(maleic acid), poly(amino acids), polyhydrocellulose, chitin, copolymers or terpolymers of the foregoing, sucrose acetate, isobutyrate, PLGA, stearic acid/N-methylpyrrolidone, or any combination of the foregoing.

10. The method of claim 1 wherein said liquid release medium has a pH, ionic strength, buffer capacity and/or temperature similar to an in vivo injection site.

11. The method of claim 10 wherein said pH is about 7.4 and said temperature is about 37° C.

12. The method of claim 10 wherein said liquid release medium comprises a physiological buffer.

13. The method of claim 12 wherein said physiological buffer further comprises gel or albumin.

14. The method of claim 1 wherein said in vivo pharmacokinetic parameter predicted is Cₘₐₓ or depot level or both.

15. A non-sink in vitro method for predicting in vivo pharmacokinetics of a depot formulation containing a poorly soluble drug compound which comprises:
a) contacting said depot formulation with a liquid release medium comprising a physiological buffer having a pH of about 7.4 at a temperature of about 37° C. under conditions effective to form a precipitate and a supernatant;
b) determining the concentration of said drug compound in said supernatant;
c) correlating said concentration to Cₘₐₓ or depot level to predict same in vivo.
